(19) United States
(12) Plant Patent Application Publication
(10) Pub. No.: US 2002/0148010 P1
(43) Pub. Date: Oct. 10, 2002

(54) Miniature rose variety 'POULgo005'

(76) Inventors: L. Pernille Olesen, Fredensborg (DK); Mogens N. Olesen, Fredensborg (DK)

Correspondence Address: Mogens N. Olesen, Poulsen Roser Pacific, Inc., 2550 Old Military Road, Central Point, OR, 97502, US

(21) Appl. No.: 09/776,450

(22) Filed: Feb. 2, 2001

(51) Int. Cl. A01H 5/00

(52) U.S. Cl. PLT/118

(57) ABSTRACT

A new miniature rose plant which has abundant, yellow flowers and attractive foliage. This new and distinct variety has shown to be uniform and stable in the resulting generations from asexual propagation.

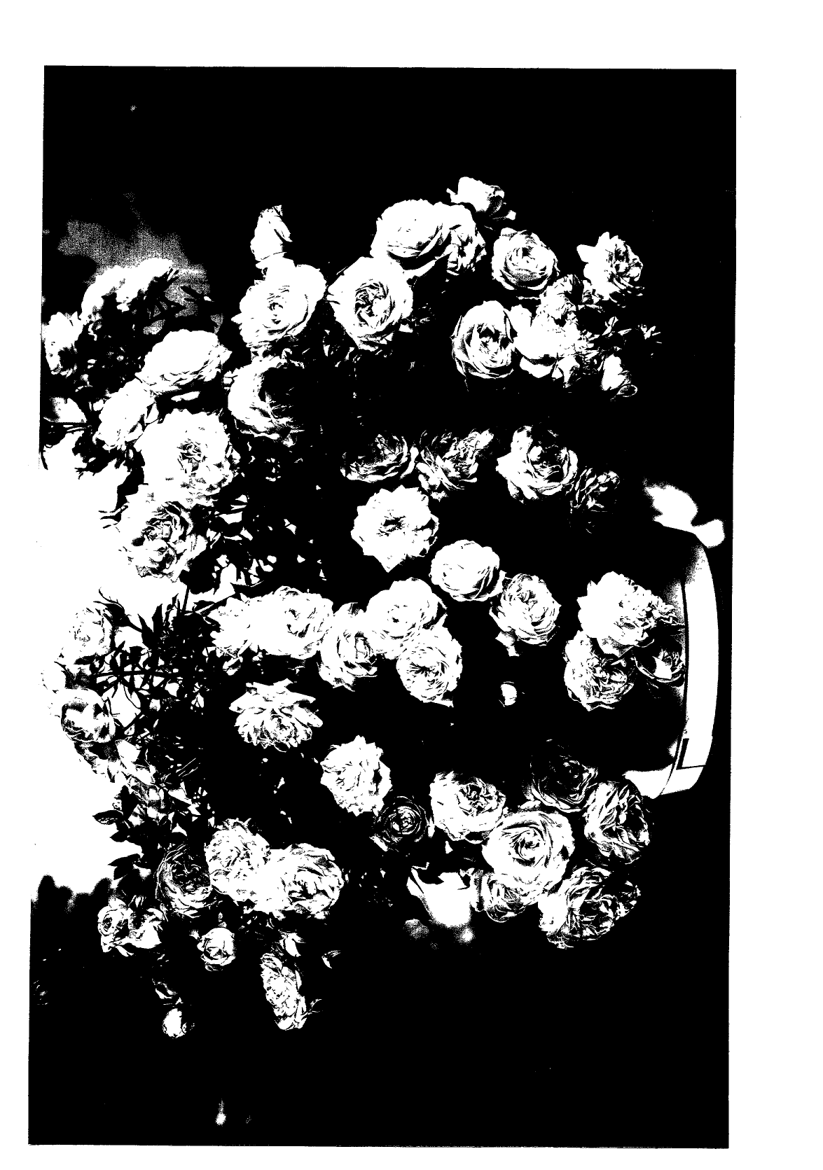

### SUMMARY OF THE INVENTION

The present invention constitutes a new and distinct variety of miniature rose plant which originated from a controlled crossing between ‘POULgelb’, described and illustrated in U.S. Plant Pat. No. 9,401, issued on Dec. 19, 1995 and an unnamed and unpatented seedling. The two parents were crossed during the summer of 1995 and the resulting seeds were planted in a controlled environment in Fredensborg, Denmark. The new variety is named ‘POULgo005’.

The new rose may be distinguished from its seed parent, ‘POULgelb’, by the following combination of characteristics:

1. ‘POULgelb’ is a miniature rose, whereas ‘POULgo005’ is a compact floribunda rose.

2. ‘POULgelb’ has deeper yellow blooms than ‘POULgo005’.

3. The blooms of ‘POULgelb’ are smaller than those of ‘POULgo005’.

The new variety may be distinguished from its pollen parent, an unnamed seedling, by the following combination of characteristics:

1. ‘POULgo005’ is a yellow rose, where the unnamed pollen parent is pink.

2. The blooms of the unnamed pollen parent are larger than those of ‘POULgo005’.

The objective of the hybridization of this rose variety was to create a new and distinct variety for garden use with unique qualities, such as:

1. Uniform and abundant flowers;

2. Vigorous, but compact growth when propagated by cuttings;

3. Disease resistance;

4. A miniature rose suitable for use in hanging basket containers in the nursery industry.

This combination of qualities is not present in previously available commercial cultivars of this type and distinguish ‘POULgo005’ from all other varieties of which we are aware.

As part of their rose development program, L. Pernille Olesen and Mogens N. Olesen germinated the seeds from the aforementioned hybridization during winter of 1995 and conducted evaluations on the resulting seedlings in a controlled environment in Fredensborg, Denmark. ‘POULgo005’ was selected in the spring of 1996 by the inventors as a single plant from the progeny of the aforementioned hybridization.

Asexual reproduction of ‘POULgo005’ by traditional budding and rooted cuttings was first done by L. Pernille and Mogens N. Olesen in their nursery in Fredensborg, Denmark in July 1996. This initial and other subsequent asexual propagations conducted in controlled environments have demonstrated that the characteristics of ‘POULgo005’ are true to type and are transmitted from one generation to the next.

### BRIEF DESCRIPTION OF THE DRAWING

The accompanying color illustration shows as true as is reasonably possible to obtain in color photographs of this type the variety rose ‘POULgo005’. Specifically illustrated in SHEET 1 is a plant of ‘POULgo005’ growing in a 20-cm hanging basket.

### DETAILED DESCRIPTION OF THE VARIETY

The following is a description of ‘POULgo005’, as observed in its growth during trials at the testing station Prüfstelle Rethmar under the testing authority Bundessortenamtin in Hannover, Germany and at the inventor's nursery in Fredensborg, Denmark. Color references are made using the Royal Horticultural Society (London, England) Colour Chart, 1995, except where common terms of color are used.

For a comparison, several physical characteristics of the rose variety ‘POULgelb’, a rose variety from the same inventors described and illustrated in U.S. Plant Pat. No. 9,401 and issued on Dec. 19, 1995 are compared to ‘POULgo005’ in Chart 1.

CHART 1

| | ‘POULgo005’ | ‘POULgelb’ |
|---|---|---|
| Color of open | Yellow Group 6D | Yellow Group 13C |
| flower, outer | | |
| side, middle | | |
| zone. | | |
| Petalage | Single | Double |
| Average Bloom | ‘Small’ | ‘Small’ |
| Diameter | | |

Parents:

Seed parent.—‘POULgelb’.

Pollen parent.—An unnamed seedling.

Classification:

Botanical.—Rosa hybrida.

Commercial.—Miniature.

### FLOWER AND FLOWER BUD

Blooming habit: Continuous

Flower bud:

Bud form.—Ovate.

Sepals.—Medium.

Receptacle.—Size: Small to medium.

Peduncle.—Surface: Very few hairs and prickles.

Borne.—Few flowering shoots.

Flower bloom:

Fragrance.—Weak.

Size.—Small.

Form.—Shape of flower when viewed from the side: Upper part: Flattened convex. Lower part: Flattened convex. Viewed from above: Irregularly rounded.

Petalage.—Single.

Color:

Petals.—Inner Side: Middle zone: Yellow Group 7B. Marginal zone: Yellow Group 7B. Basal petal spot: None observed. Outer side: Middle zone: Yellow Group 7B. Marginal zone: Yellow Group 7B. Basal petal spot: None observed.

General Tonality: On open flower, Yellow Group 7B. No change in the general tonality at the end of the 10th day.

Petals:

Petal Reflex.—Weak.

Undulation of margin.—Weak.

Size.—Small.

Reproductive Organs:

Filaments.—Color: Yellow

Hips.—Shape: Pitcher-shaped.

### PLANT

Plant growth: Spreading. Procumbent to arching. Height: 40 to 60 cm.

Stems:

Thorns.—Incidence: Many short prickles and few long prickles. Size: Small. Shape: Linear to concave.

Plant foliage:

Leaf size.—Small to medium.

Glossy.—Medium glossiness.

Color.—Upper Leaf Surface: Dark green Juvenile foliage: Exhibits anthocyanin on young shoots. Anthocyanin intonation: Color: Reddish brown.

Plant leaflets:

Leaflet.—Cross Section: Slightly concave. Margin Undulation: Weak.

Terminal leaflet.—Length of blade: Short. Width of blade: Narrow to medium. Shape: Rounded.

Disease resistance: Above average resistance to mildew, rust, black spot, and Botrytis under normal growing conditions in Hannover, Germany and Fredensborg, Denmark.

Cold Hardiness: The variety ‘POULgo005’ has been found to be cold hardy in Fredensborg, Denmark and Hannover, Germany.

1. A new and distinct variety of rose plant of the miniature rose class, substantially as herein illustrated and described as a distinct and novel rose variety due to its abundant yellow flowers, procumbent to arching habit, disease resistance, and extended period of bloom.
